(19) United States
(12) Patent Application Publication
(10) Pub. No.: US 2024/0423965 A1
(43) Pub. Date: Dec. 26, 2024

(54) FIXED DOSE COMBINATION OF CHOLINESTERASE INHIBITOR AND A QUATERNARY AMMONIUM ANTIMUSCARINIC AGENT TO TREAT NEURODEGENERATIVE COGNITIVE DISORDERS

(71) Applicant: QAAM PHARMACEUTICALS, LLC, Canandaigua, NY (US)

(72) Inventor: Steven A. Rich, Canandaigua, NY (US)

(21) Appl. No.: 18/826,565

(22) Filed: Sep. 6, 2024

Related U.S. Application Data
(62) Division of application No. 17/163,010, filed on Jan. 29, 2021, now Pat. No. 12,109,202.

(51) Int. Cl. A61K 31/438; A61K 9/00; A61K 9/48; A61K 9/70; A61K 31/165; A61K 31/4015; A61P 25/28

(52) U.S. Cl. CPC A61K 31/438; A61K 9/0053; A61K 9/4825; A61K 9/4866; A61K 9/7023; A61K 31/165; A61K 31/4015; A61P 25/28

(57) ABSTRACT

A method for improving cognitive function in a human suffering from a neurodegenerative cognitive disorder includes introducing a therapeutic amount of quaternary ammonium antimuscarinic compound into a body of a human at a first initial time, introducing a therapeutic amount of a cholinesterase inhibitor into the body of the human at a second initial time, releasing the therapeutic amount of the quaternary ammonium antimuscarinic compound into the body of the human at a first rate, and releasing the therapeutic amount of the cholinesterase inhibitor into the body of the human at a second rate. The first rate is different than the second rate.

### CROSS-REFERENCE TO RELATED APPLICATIONS

This application is a divisional of U.S. patent application Ser. No. 17/163,010, filed on Jan. 29, 2021, the disclosure of which is incorporated by reference.

### FIELD OF THE INVENTION

The invention pertains to the field of cognitive treatments. More particularly, the invention pertains to improvements in cognitive function in patients treated with cholinesterase inhibitors in combination with quaternary ammonium anti-cholinergic muscarinic receptor antagonist.

### BACKGROUND OF THE INVENTION

Neurodegenerative cognitive diseases impart costs on individuals and society in terms of loss of function, costs of care, and the personal loss of identity associated with pain and suffering. Such diseases or neurodegenerative cognitive disorders include, but are not limited to Alzheimer's Disease, Dementia with Lewy Body, Parkinson's Disease, and Progressive Supranuclear Palsy.

Cognitive neurodegeneration is a serious neurological condition that is very common in the elderly. By estimate, approximately one-third of people who live to be over 80 years of age will be diagnosed with some form of neurodegenerative cognitive disorder. Cognitive neurodegeneration can result from a variety of disease processes, such as, but not limited to:

### Neurodegenerative Dementia

Alzheimer's Disease
Pick's Disease
Progressive Supranuclear Palsy
Dementia with Lewy Bodies
Parkinson's Disease
Fronto-temporal Dementia

### Vascular Diseases

Stroke
Multi-infarct dementia
Subarachnoid hemorrhage

### Head Trauma

### Infections

Post-encephalitic dementia
Syphilis
Herpetic encephalitis

### Congenital Abnormalities

Trisomy 21

### Toxic Brain Injuries

Wernike Encephalopathy
Krorsakoff psychosis
Alcoholic amnesic syndrome
Alcoholic dementia

The primary result of this general condition is a universal decline in the intellectual function of the individual, usually resulting in significant impediments to normal daily functions. While there is currently no disease modifying treatment available for most forms of cognitive neurodegeneration, certain treatments are available to alleviate the symptoms and improve cognitive functioning to varying degrees, which can at least delay the need for institutionalizing these individuals.

It has been clinically determined that the decline of the neurotransmitter chemical acetylcholine in the brain is one of the primary mechanisms of declining mental function. Medications that can prevent or at least minimize the breakdown of acetylcholine in the brain provide significant improvement in the cognitive abilities of patients diagnosed with neurodegenerative cognitive disorders. These medications are commonly referred to as acetyl-cholinesterase inhibitors. However, as with any medication, there are side effects corresponding with administration of acetylcholinesterase inhibitors. For example, acetyl-cholinesterase inhibitors exacerbate urinary and fecal incontinence in patients administered these drugs. Other side effects include a reduced heart rate, sweating, vasodilation and increased bronchial secretions. Such side effects may be so uncomfortable for many elderly patients that the patient is unable to tolerate effective dosing of acetyl-cholinesterase inhibitors to successfully treat the neurodegenerative cognitive disorders.

Attempts to ameliorate these undesirable side effects in patients with cognitive neurodegeneration include the administration of, for example, antimuscarinic anti-cholinergic drugs (commonly called “anti-muscarinics”). These drugs block the peripheral stimulation of the acetylcholine receptors. Unfortunately, the use of these medications to treat the side effects of acetyl-cholinesterase inhibitors, previously mentioned, often contribute to cognitive neurodegeneration, which is what the acetyl-cholinesterase inhibitor is intended to treat in the first place. Thus, the benefits of using these drugs must be balanced with the risks of exacerbating the existing neurodegenerative cognitive disorder. As a result, many patients are either inadequately treated or go untreated.

Certain developmental disorders, particularly the Autism Spectrum Disorders, may share a neurodegenerative process, as suggested by the observed fact that normal development is often interfered with at some point after the first year of age. Increasing data suggests that neurodegenerative processes may reflect impaired clearance of endogenous molecules from the central nervous system. Suspected molecules include, but are not limited to, beta amyloid, alpha synuclein, tau, and numerous modifications of these molecules that occur through endogenous metabolic processes.

Despite the development of technologies for early detection of neurodegenerative processes, such as radio labeled antibodies against amyloid, the clinical role of such interventions has been limited by the fact that the justification for the cost of these diagnostic interventions is difficult in the absence of disease modifying therapies.

Over the years, researchers have studied the cognitive effects of anti-cholinergic drugs and have found that anti-cholinergic and anti-muscarinic drugs cause cognitive decline in the elderly and further cognitive decline in those already impaired. This effect is particularly true of drugs to treat urinary incontinence and overactive bladder. In fact, many practitioners refer to the dilemma regarding treatment of neurodegenerative cognitive disorders in combination with incontinence as choosing between “your brain versus your bladder”. When prescribing medications, a practitioner has to work with their patients and their families and often decide whether to treat the incontinence caused by the medications being used to try to improve cognitive function, which would result in further detriment of cognitive function, or instead rely on alternative solutions to incontinence issues (including the often embarrassing use of adult diapers, or even institutionalization of the incontinent patient).

One severe type of cognitive impairment that often afflicts the elderly is referred to as acute delirium. The primary indicators are a pronounced change in mental status that rapidly fluctuates, the inability to maintain normal degrees of attention, disorganized thinking, and vacillating levels of consciousness. Acute delirium can often result from a severe medical illness, recent surgery, and use of several medications or interactions between various medications. The impact of acute delirium on patients is severe and often chronic, frequently leading to death.

While the neurological mechanism by which acute delirium occurs is not completely understood, like with other neurodegenerative cognitive disorders, the neurotransmitter acetylcholine is thought to play a significant role. In patients suffering from dementia, a decline in acetylcholine has been seen in post mortem studies. As with treatments for neurodegenerative cognitive disorders, the use of cholinesterase inhibiting medications has been determined to prevent, to varying degrees, the breakdown of acetylcholine in the brain. The undesirable side effects discussed above, however, outside the central nervous system (CNS), often result. In order to minimize these problems, the administration of drugs that block the peripheral system effects of cholinesterase inhibitors would be desirable. Unfortunately, in a manner similar to other neurodegenerative cognitive disorders, anti-cholinergics frequently contribute to the underlying problem by causing central nervous system toxicity.

### SUMMARY OF THE INVENTION

A method of treating neurodegenerative cognitive disorders using cholinesterase inhibiting medications reduces or avoids the undesirable side effects discussed above.

According to an embodiment, a method for improving cognitive function in a human suffering from a neurodegenerative cognitive disorder includes administering to a patient a total daily dose of a cholinesterase inhibitor and a quaternary ammonium antimuscarinic compound. The total daily dose is divided into one or more doses. The quaternary ammonium antimuscarinic compound is administered in a first drug delivery element configured for absorption into a body of the patient at a first rate, and the cholinesterase inhibitor is administered in a second drug delivery element configured for absorption into the body of the patient at a second rate, the first rate being quicker than the second rate.

In an aspect of this embodiment, the one or more doses are administered at least one hour before the patient eats.

According to another embodiment, a method for improving cognitive function in a human suffering from a neurodegenerative cognitive disorder includes introducing a therapeutic amount of 2-8 mg of a quaternary ammonium antimuscarinic compound into a body of a human at a first initial time, introducing a therapeutic amount of 3-48 mg of a cholinesterase inhibitor into the body of the human at a second initial time, releasing the therapeutic amount of 2-8 mg of the quaternary ammonium antimuscarinic compound into the body of the human at a first rate, and releasing the therapeutic amount of 3-48 mg of the cholinesterase inhibitor into the body of the human at a second rate. The first initial time is the same as the second initial time and the first rate is different than the second rate.

### BRIEF DESCRIPTION OF THE DRAWING

FIG. 1 shows an embodiment of a drug delivery element.

FIG. 2A shows a graph illustrating the results of a Phase 1 clinical trial of rivastigmine and glycopyrrolate administrations.

FIG. 2B shows a graph illustrating the results of a Phase 1 trial with rivastigmine and trospium administrations.

FIG. 3 shows a graph depicting a rivastigmine blood serum concentration time profile of a patient after ingesting the co-formulation of rivastigmine/glycopyrrolate taken one hour before eating food.

FIG. 4 shows a bar graph displaying a rivastigmine steady state blood serum concentration level of patients afflicted with Dementia with Lewy bodies, wherein the patients have shown disease-modifying effects.

### DETAILED DESCRIPTION OF THE INVENTION

Patients suffering from various forms of neurodegenerative cognitive disorders, including acute delirium, can be treated with an effective amount of medication to reduce, minimize, or entirely alleviate these conditions without imposing upon the patients the undesired peripheral effects of urinary and/or fecal incontinence, nausea, bradychardia, bronchorrhea, and/or bronchospasm, which often coexist with these neurodegenerative cognitive disorders. The treatment can alter the disease progression of progressive dementias and other neurodegenerative cognitive disorders. The desired goal of treatment is to administer the most efficacious type and quantity of medication to treat the neurological condition without increasing the unwanted side effects of high doses of those medications. Appropriate treatment includes quaternary ammonium anti-cholinergic muscarinic receptor antagonists administered, at adequate levels, in combination with cholinesterase inhibitors, at doses that minimize the adverse effects inherent to unmitigated high doses of cholinesterase inhibitors that are used to treat neurodegenerative cognitive disorders. The treatment results in a modification of the disease, namely a slowing of the disease progression. In an embodiment, the quaternary ammonium anti-cholinergic muscarinic receptor antagonist administered to ameliorate the disease is glycopyrrolate and the acetyl-cholinesterase inhibitor administered to ameliorate the disease is rivastigmine. Rivastigmine blood serum concentrations between 0-25 ng/ml (nanograms per milliliter) are medically associated with improved symptom control in several neurodegenerative diseases. Generally, rivastigmine blood serum concentrations greater than 25 ng/ml are associated with the aforementioned adverse effects including urinary and/or fecal incontinence, nausea, bradycardia, bronchorrea, bronchospasm, and orthostatic hypotension.

The quaternary ammonium anti-cholinergic muscarinic receptor antagonist prevents, or substantially ameliorates, the undesirable side effects of acetyl-cholinesterase inhibitors, which permits the administration of higher doses of acetyl-cholinesterase inhibitors than patients could otherwise tolerate. Quaternary ammonium anti-cholinergic muscarinic receptor antagonists can be administered to patients suffering from neurodegenerative cognitive disorders, in general, such as dementia, acute dementia and dementia with Lewy Bodies, because such compounds are excluded from the central nervous system. The patients can also be administered the cholinesterase inhibitor rivastigmine. Such treatment results in a marked improvement in cognitive function, in addition to an improvement in controlling the adverse effects of excessive acetylcholine including, but not limited to, urinary and/or fecal incontinence, nausea, bradychardia, bronchorrhea, and bronchospasm.

A benefit of using the anti-cholinergic muscarinic agents of the methods and compositions described herein is that the maximum dosing of the acetyl-cholinesterase inhibitor to effectively treat the neurodegenerative cognitive disorder afflicting the patient can be administered and maintained. Higher doses of acetyl-cholinesterase inhibitors may slow, or stop, the degenerative process through mechanisms that include improved clearance of toxic molecules. The use of the co-formulation of quaternary ammonium anti-cholinergic muscarinic receptor antagonist and cholinesterase inhibitors permits the administration of therapeutic dosages of cholinesterase inhibitors, thus maximizing the beneficial effect of the therapeutic drugs.

Suitable quaternary ammonium anti-cholinergic muscarinic receptor antagonists include the drugs trospium and glycopyrrolate, but not all antimuscarinic drugs are the same. One method of differentiating the various drugs in this category is by lipid solubility. It has been determined that quaternary ammonium compounds of the class of anti-cholinergic muscarinic agents having very low lipid solubility are desirable for use within the context of the methods and compounds described herein. As a result of their low lipophilicity (the ability of a compound to dissolve in a lipid medium), these molecules tend not to cross the blood/brain barrier as readily as those having higher lipid solubility. By not crossing the blood-brain barrier, these compounds do not interfere with the normal function of acetylcholine in the central nervous system, nor do they interfere with the beneficial effects of acetyl-cholinergic inhibitors for the treatment of neurodegenerative cognitive disorders. Further, these low lipid solubility quaternary ammonium anti-cholinergic muscarinic receptor antagonist drugs ameliorate the undesired peripheral effects from the use of acetyl-cholinesterase inhibitors, such as urinary and/or fecal incontinence, nausea, bradychardia, bronchorrhea, and bronchospasm.

The quaternary ammonium anti-cholinergic muscarinic agents for use in the methods described herein can include trospium and glycopyrrolate (non-quaternary anti-cholinergic agents include, but are not limited to, oxybutynin, tolterodine, darifenacin and solefenacin). Log P, a recognized parameter proportional to octanol/water partitioning coefficient, is a standard for measuring comparative solubility of a compound in a lipid compared to water. The comparative solubility of a drug molecule in water is the most important physical property that correlates with the capacity of the drug molecule to cross the blood/brain barrier and to interfere with the normal functioning of acetylcholine in the central nervous system. A low log P value represents low lipid solubility and low probability of crossing the blood/brain barrier. Both glycopyrrolate and trospium have a low log P based upon the nature of the chemical structure of glycopyrrolate and trospium. Comparatively, the standard anti-muscarinic drugs that are currently in use have a log P value as high as 6.076 (tolterodine). Trospium has a log P value of 0.78 and the calculated lipophilicity of glycopyrrolate is −75.75, thus making glycopyrrolate and trospium preferred compounds to achieve the therapeutic goals stated previously within the context of the embodiments described herein.

Glycopyrrolate (also known as glycopyrronium bromide) is a bromide salt with a quaternary ammonium counterion with the chemical name of 3-[cyclopentyl(hydroxy)phenylacetoxy]-1,1-dimethyl pyrrolidinium bromide, a molecular formula of C₁₉H₂₈BrNO₃ and a molecular weight of 398.34.

Trospium (also known as trospium chloride) is a quaternary ammonium salt with the chemical name of 3 (2 hydroxy-2,2 diphenylacetoxy)spiro[bicyclo[3.2.1] octane-8,1′ pyrrolidin]-1′-ium chloride. The molecular formula of trospium chloride is C₂₅H₃₀ClNO₃ and its molecular weight is 427.97. The chemical structure of trospium chloride is:

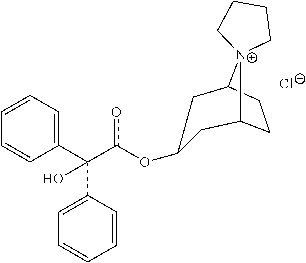

The quaternary ammonium anticholinergic muscarinic agents may be administered concurrently with any of the various acetyl-cholinesterase inhibitors used to treat neurodegenerative cognitive disorders. Such drugs include, but are not limited to:
donepezil
rivastigmine
galantamine
tacrine
physostigmine
pyridostigmine
neostigmine
ipidacrine
phenserine
icopezil
zanapezil
ambenonium
edrophonium
huperzine A
ladostigil

In order to effectively treat patients suffering from neurodegenerative cognitive disorders that exhibit the unwanted adverse effects of excessive acetylcholine from a cholinesterase inhibitor including, but not limited to, urinary and/or fecal incontinence, nausea, bradycardia, bronchorrea, bronchospasm, and orthostatic hypotension, quaternary ammonium anti-muscarinic agents can be combined with suitable acetyl-cholinesterase inhibitors. Both classes of drugs can be administered intravenously, intramuscularly (“parenterally”), or transdermally. Because patients with acute delirium are often confused and/or belligerent, these patients often refuse to take oral medications. In some cases, however, oral administration may be successfully achieved.

The quaternary ammonium antimuscarinic drugs (QAAM) provide benefits because of the drugs' ability to antagonize endogenous acetylcholine during periods of excessive acetylcholine production, or prolonged acetylcholine affect from physiologic and pharmacologic reasons. These compounds share the property that they do not appreciably penetrate the central nervous system (CNS), and glycopyrrolate bromide and trospium chloride have been particularly useful in treating patients in need of a peripheral anticholinergic effect on antimuscarinic receptors.

In patients with neurodegenerative cognitive disorders being treated with ACEI-QAAM, higher doses of all the acetylcholinesterase inhibitors are tolerated at least four times the usual monotherapy dose. For rivastigmine, the maximum monotherapy dose in the US is currently 12 mg/day orally or 9.5 mg/day transdermally (the FDA recently approved the use of 13.5 mg/day transdermally, but the maximum orally approved dose is still 12 mg/day). In some embodiments of the present invention, dosages up to 48 mg/day orally are used. In one embodiment, the daily dosage of the combination treatment is rivastigmine at 24-48 mg/day (or 17.9-39.9 mg transdermally) in combination with 1-2 mg of glycopyrrolate, orally, twice daily.

For patients exhibiting symptoms of dementia with Lewy bodies, a combination of rivastigmine and glycopyrrolate, for example, can be administered. In one embodiment, 12 mg to 48 mg/day of rivastigmine is administered orally, or 17.9 mg to 39.9 mg is administered transdermally. In another embodiment, 19 mg to 24 mg/day of rivastigmine is administered. In yet another embodiment, a minimum of 24 mg/day of rivastigmine is administered. In other embodiments, dosages of up to 48 mg/day orally of rivastigmine are used. In one embodiment, glycopyrrolate can be administered at the rate of 2 to 8 mg/day.

Generally, rivastigmine blood serum concentrations greater than 25 ng/ml are associated with the aforementioned adverse effects including urinary and/or fecal incontinence, nausea, bradycardia, bronchorrhea, bronchospasm, and orthostatic hypotension. Levels of this magnitude or greater may also be associated with more symptomatic improvement and disease modifying effects.

Administering quaternary ammonium antimuscarinic agents on an empty stomach can facilitate bioavailability. Comparatively, administering cholinesterase inhibitors, such as rivastigmine, with food can help delay the absorption of, and reduce the peak concentration of the cholinesterase inhibitors, which is associated with the previously mentioned adverse effects such as urinary and/or fecal incontinence, nausea, bradycardia, bronchorrhea, bronchospasm, and orthostatic hypotension. Accordingly, the coordinated absorption of quaternary ammonium antimuscarinic agents with cholinesterase inhibitors can help reduce or avoid the adverse effects of the cholinesterase inhibitors.

Taking the two drugs at separate time intervals related to food ingestion is inconvenient for patients suffering from neurodegenerative cognitive disorders. The separate administration of the quaternary ammonium antimuscarinic compound and the cholinesterase inhibitor to patient populations that present adherence difficulty, especially coordinated around food ingestion, can also be problematic. As previously discussed, patients with acute delirium are often confused and/or belligerent, such that these patients often refuse to take oral medications or forget. Simplifying oral dosing or the number of doses is beneficial in these instances. Coformulating glycopyrrolate and rivastigmine in a single dose yields greater case of administration because separate administration of the two medications to patient populations that present adherence difficulty is minimized. The coformulation can be administered to the patient transdermally, in addition to orally or by other methods of administering. Transdermal administration, for example, can be easier with uncooperative or forgetful patients than oral administration.

In an embodiment, a method of improving cognitive function in a human suffering from a neurodegenerative cognitive disorder includes administering, orally, to a patient, a total daily dose of 3-48 mg of a cholinesterase inhibitor (rivastigmine), at a first initial time and at a first rate, and 2-8 mg of a quaternary ammonium antimuscarinic compound (glycopyrrolate) at a second initial time and at a second rate and the first initial time is different from the second initial time, and the first rate is different than the second rate. The total daily dose can be divided into individual doses administered at least one hour before food intake.

The total daily dose can be administered in one or more drug delivery elements that each release a therapeutic amount of 3-48 mg of a cholinesterase inhibitor, such as rivastigmine, into the human body and a therapeutic amount of 2-8 mg of a quaternary ammonium antimuscarinic compound, such as glycopyrrolate or 20-60 mg of trospium. Various forms of the drug delivery element can include a transdermal patch, a tablet, an injectable liquid, and a pill, tablet, or capsule. The drug delivery element can include these, as appropriate, or any other now-known or future-developed drug delivery structure.

According to methods of the invention, the cholinesterase inhibitor and the quaternary ammonium antimuscarinic compound can be administered in separate drug delivery elements, or as a single drug delivery element with multiple portions. If administered separately, according to some embodiments, the therapeutic amount of the quaternary ammonium anti-cholinergic muscarinic receptor antagonist can be administered to the patient orally, intravenously, or intramuscularly and the therapeutic amount of the acetyl-cholinesterase inhibitor can be administered to the patient either orally or transdermally. The cholinesterase inhibitor can be administered to the patient at least one hour before food intake.

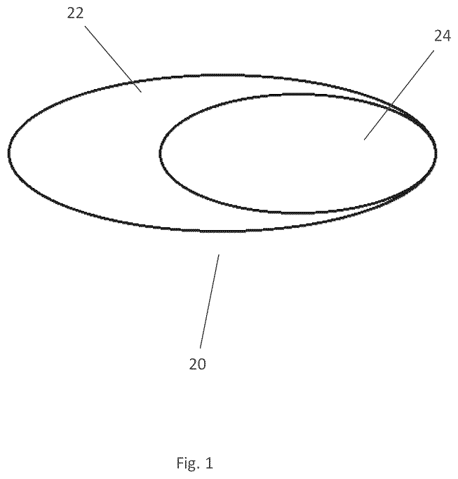

FIG. 1 schematically illustrates a single drug delivery element 20 including a first portion 22 and a second portion 24 attached to or enclosed within the first portion 22. The first portion 22 can include quaternary ammonium antimuscarinic compound and the second portion 24 can include cholinesterase inhibitor. The first portion 22 can be rapidly dissolving, such as but not limited to a rapidly dissolving capsule (e.g., a gelatin capsule), and the second portion 24 can be an enclosed acid-resistant structure, such as but not limited to a capsule, configured for a delayed release. A hydroxypropylmethylcellulose (HPMC) capsule is an example of an enclosed acid-resistant structure. The delayed release of the second portion 24 can facilitate cholinesterase inhibitor acting on the small intestine.

In an embodiment, the drug delivery element 20 can be administered at least one hour before food intake. After administration, in some embodiments, the first portion 22 and the second portion 24 begin to dissolve concurrently. In these embodiments, though, the first portion 22 releases a therapeutic amount of 2-8 mg of a quaternary ammonium antimuscarinic compound into the human body according to a first dissolve rate, while the second portion 24 releases a therapeutic amount of 3-48 mg of a cholinesterase inhibitor into the human body according to a second dissolve rate. The first dissolve rate is faster than the second dissolve rate. The first portion 22 including a QAAM compound dissolves relatively quickly, while the second portion 24 including a cholinesterase component dissolves more slowly than the first portion 22. Thus, the slower dissolution of the second portion 24 enables the second portion 24 to be released after the patient has eaten, and the patient's body has absorbed, food.

In some embodiments, such as when the second portion 24 is enclosed within the first portion 22, the onset of dissolution of the second portion 24 is delayed from the onset of dissolution of the first embodiment 22. In this situation, the term “dissolve rate”, as used herein, is an average dissolve rate calculated from the onset of dissolution of the first portion 22 or from the introduction of the drug delivery element 20 into the human body. In comparison, the term “instant dissolve rate” is intended to mean the dissolve rate at any given point in time. Similarly, the term “absorption rate” when applied to the QAAM compound and the cholinesterase inhibitor contained in the drug delivery element 20 is intended to mean an average rate of absorption calculated from the same point in time for both the QAAM compound and the cholinesterase inhibitor, such as the time the drug delivery element 20 is introduced into the human body or the time when the QAAM compound begins to be absorbed by the human body. The term “instant absorption rate” is intended to mean the absorption rate at any given point in time. Accordingly, whether the second portion 24 begins to dissolve later than the first portion 22 and then finishes dissolving later also, or whether the second portion 24 begins to dissolve at the same time as the first portion 22 but dissolves with a slower instant dissolve rate, such that it finishes dissolving later, the second portion 24 can be considered to have a slower dissolve rate than the first portion 22.

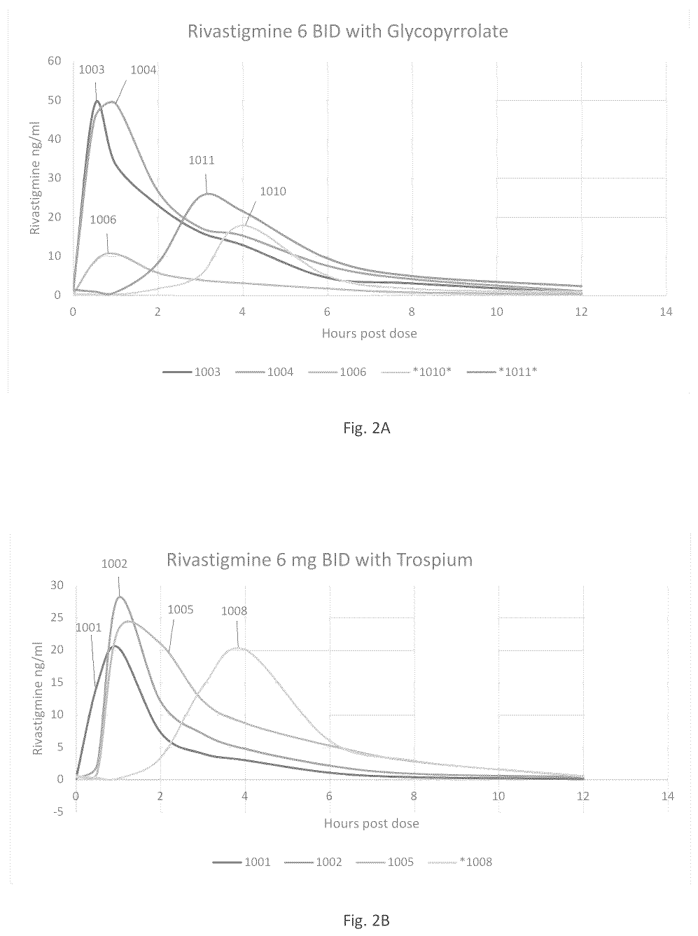

Patients treated with acetyl-cholinesterase inhibitors in combination with quaternary ammonium anti-cholinergic muscarinic agents (ACEI-QAAM) can be studied to collect data on the cognitive, affective, and functional improvement over time. Positive experimental data and results show delayed and reduced peak rivastigmine blood serum levels in patients using the method of the present invention, as reflected by the data displayed in FIG. 2A through FIG. 4.

FIG. 2A compares rivastigmine blood serum concentration time profiles 12 hours post dose of five subjects: (a) two normal control subjects that were administered immediate release glycopyrrolate and increasing doses of rivastigmine simultaneously in a fasted state one hour prior to any food consumption; (b) two normal control subjects that were administered glycopyrrolate and rivastigmine separately with the glycopyrrolate administered while the subjects were in a fasting state, and with the rivastigmine administered to the subjects with food after one hour; and (c) a test subject that was administered the new co-formulated dosage of rivastigmine and glycopyrrolate one hour before consuming a meal.

In a Phase 1 trial, as shown in FIG. 2A, patient 1003 and patient 1004 were both normal control subjects that were administered immediate-release glycopyrrolate and increasing doses of rivastigmine, simultaneously, in a fasted state one hour prior to eating. Patient 1010 and Patient 1011 were administered the two medications separately with glycopyrrolate being administered in a fasting state and rivastigmine being administered with food approximately one hour later in order to ameliorate some of the side effects such as nausea. Patient 1006 was administered a lower dose of rivastigmine with glycopyrrolate simultaneously in a fasting state. The serum rivastigmine levels were obtained over the course of twelve hours post-administration. The results shown in FIG. 2A show that the high rivastigmine levels associated with adverse effects of the cholinesterase inhibitor, rivastigmine, were avoided with the giving the glycopyrrolate and rivastigmine doses separately in fasting and fed states respectively, 1 hour apart, and resulting coordinated absorption, of the quaternary ammonium anti-cholinergic muscarinic receptor antagonist, glycopyrrolate, as compared to the coadministration, in patients 1003, and 1004, of glycopyrrolate and rivastigmine in a fasted state. Comparatively, the separate administration of glycopyrrolate, while the patients 1010 and 1011 were in a fasted state, and rivastigmine, and while the patients were fed, was attenuated, showing the delayed peak serum concentration associated with lower peak rivastigmine levels and reduced adverse effects, particularly nausea. It was proposed that with the coformulation, patients can take one dose of the medication in a single drug delivery element and eliminate the separate administration of two medications to patient populations that present adherence difficulty. The coformulation allows this singular does and still approximates the rivastigmine blood serum concentrations seen with separate administration of the components in fed and fasting states. Time profile of patient 1006, 10 ng/ml, obtained with the co-formulated drug delivery element taken in a fasting state, displays absorption kinetics similar to that of the separately administered components.

FIG. 2B compares the rivastigmine blood serum concentration time profiles, in normal control subjects, obtained with: (a) concurrent administration of 6 mg rivastigmine and 20 mg trospium, a different quaternary ammonium antimuscarinic agent from glycopyrrolate in a fasting state; (b) separated administration of 6 mg rivastigmine and 20 mg trospium in a fasting state, and 6 mg of rivastigmine in a fed state one hour later; and (c) the novel co-formulated drug dosage administration simultaneously, in an embodiment of the present invention, taken (BID) twice per day. Patients 1001, 1002, and 1005 were administered trospium and rivastigmine simultaneously in a fasting state. Subject 1008 was administered trospium and rivastigmine in fasting and fed states, respectively. The rivastigmine blood serum concentrations illustrated in FIG. 2B show attenuated peak rivastigmine levels achieved by taking the medication with food, similar to that seen with glycopyrrolate and rivastigmine administered separately.

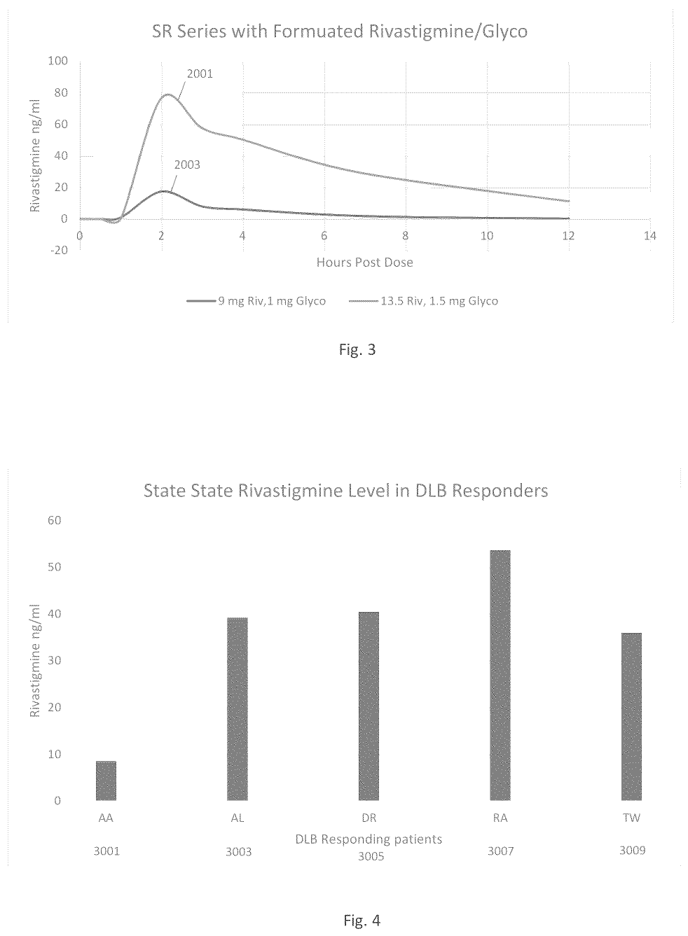

FIG. 3 displays the effects of the co-formulation of a rivastigmine/glycopyrrolate administration in a single control subject. This co-formulation was administered in the morning one hour before food was consumed on separate days in two different doses. In an embodiment, the drug delivery system is a co-formulated capsule having 0.5 mg glycopyrrolate and 4.5 mg rivastigmine. With this specific embodiment, doses can be multiples of 0.5 mg glycopyrrolate and 4.5 mg rivastigmine and can be administered two, three, or four times per day depending upon the disease state. In other embodiments, the ratio of the quaternary ammonium antimuscarinic/cholinesterase inhibitor compound my vary in order to achieve a dosing regimen optimized to the patient and the disease being treated.

FIG. 3 compared with FIGS. 2A and 2B demonstrates that the serum concentration-time profile of rivastigmine achieved with the single dosing of the compounded drug delivery system is similar to that achieved by separate administration of the components in fasting and fed states. Dosing with the co-formulated drug delivery system was not associated with the nausea associated with the simultaneous administration of both components in a fasting state.

FIG. 4 exhibits the steady state blood serum rivastigmine levels in patients 3001, 3003, 3005, 3007, 3009 that have dementia with Lewy bodies and had demonstrated a reduction in the rate of cognitive decline over years with combined rivastigmine and glycopyrrolate administered separately. These subjects did not display adverse effects despite high doses of rivastigmine—36-42 mg/day orally, and 39.9 mg/day transdermally. Patient 3009 was administered two co-formulated capsules of 0.5 mg glycopyrrolate and 4.5 mg glycopyrrolate four times per day, one hour before eating, for a daily dose of rivastigmine of 36 mg. Based upon the concentration-time curves, it can be derived that the co-formulation can maintain a similar concentration of rivastigmine throughout the dosing interval.

The constellation of symptoms in dementia with Lewy Bodies includes vivid hallucinations, falling, precipitous drops in blood pressure with standing (orthostatic hypotension). Rapid fluctuations in consciousness and orientation occur and there is a commonly a sleep disturbance (Rapid Eye Movement Sleep Behavioral Disturbance). The patients are often intolerant of drugs commonly used for psychosis (Neuroleptic Sensitivity Syndrome). The patients have variable moods with a predisposition to depression and varying degrees of symptoms similar to Parkinson's disease. The co-formulation, in a single drug delivery element, can be more conveniently administered to patients that have dementia with Lewy bodies, and can be better tolerated by these patients as well.

Dementia with Lewy Bodies is a progressive dementing disease with some features of Parkinson's disease. Dementia with Lewy Bodies progresses faster than Alzheimer's Disease and provides an opportunity to observe a change in the deterioration of neurodegenerative cognitive ability over time (Olichney JM et al. “Cognitive Decline is Faster in Lewy Body Variant than in Alzheimer's Disease”. Neurology 1998;51 (2)351, herein incorporated by reference). Cholinesterase inhibition, particularly with rivastigmine, is the most efficacious treatment for Dementia with Lewy Bodies.

Accordingly, it is to be understood that the embodiments of the invention herein described are merely illustrative of the application of the principles of the invention. Reference herein to details of the illustrated embodiments is not intended to limit the scope of the claims, which themselves recite those features regarded as essential to the invention.

What is claimed is:

1. A method for improving cognitive function in a human suffering from a neurodegenerative cognitive disorder, the method comprising:
administering to a patient a total daily dose of a cholinesterase inhibitor and a quaternary ammonium antimuscarinic compound, the total daily dose divided into one or more doses administered at least one hour before the patient eats, wherein the quaternary ammonium antimuscarinic compound is administered in a first drug delivery element configured for absorption into a body of the patient at a first rate, and the cholinesterase inhibitor is administered in a second drug delivery element configured for absorption into the body of the patient at a second rate, the first rate being quicker than the second rate.

2. The method of claim 1, wherein the first drug delivery element is attached to the second drug delivery element.

3. The method of claim 1, wherein the first drug delivery element is configured to dissolve rapidly in the stomach, and the second drug delivery element is enclosed, acid resistant, and configured for delayed release in a small intestine of the body of the patient.

4. The method of claim 1, wherein the quaternary ammonium antimuscarinic compound is glycopyrrolate.

5. The method of claim 4 wherein the glycopyrrolate is administered orally.

6. The method of claim 5, wherein the total daily dose includes 2-8 mg glycopyrrolate.

7. The method of claim 1, wherein the cholinesterase inhibitor is rivastigmine.

8. The method of claim 7, wherein the rivastigmine is administered transdermally.

9. The method of claim 7, wherein the rivastigmine is administered orally as a minimum of about 12 mg/day orally or a minimum of about 13.3 mg/day transdermally.

10. The method of claim 7, wherein the rivastigmine is administered transdermally as an amount between about 19 mg and 54 mg.

11. The method of claim 1, wherein the total daily dose includes 3-48 mg of the cholinesterase inhibitor and 2-8 mg of the quaternary ammonium antimuscarinic compound.

12. The method of claim 1, wherein the quaternary ammonium antimuscarinic compound is glycopyrrolate, the cholinesterase inhibitor is rivastigmine, and the total daily dose includes 2-4 mg/day of glycopyrrolate and 12-48 mg/day of rivastigmine.

13. The method of claim 1, wherein at least one of the first drug delivery element and the second drug delivery element is a capsule.

14. The method of claim 1, wherein the cholinesterase inhibitor is selected from the group consisting of donepezil, rivastigmine, galantamine, tacrine, physostigmine, pyridostigmine, neostigmine, ipidacrine, phenserine, icopezil, zanapezil, Ambenonium, Edrophonium, Huperzine A, and Ladostigil.

15. The method of claim 1, wherein the quaternary ammonium antimuscarinic compound is trospium.

16. The method of claim 15, wherein the total daily dose of trospium is 20-60 mg.

17. A method for improving cognitive function in a human suffering from a neurodegenerative cognitive disorder, the method comprising:
introducing a therapeutic amount of 2-8 mg of a quaternary ammonium antimuscarinic compound into a body of a human at a first initial time;
introducing a therapeutic amount of 3-48 mg of a cholinesterase inhibitor into the body of the human at a second initial time;
releasing the therapeutic amount of 2-8 mg of the quaternary ammonium antimuscarinic compound into the body of the human at a first rate; and
releasing the therapeutic amount of 3-48 mg of the cholinesterase inhibitor into the body of the human at a second rate,
wherein the first initial time is the same as the second initial time and the first rate is different than the second rate.

18. The method of claim 17, wherein introducing the therapeutic amount of the quaternary ammonium anti-cholinergic muscarinic receptor antagonist to the human occurs orally, intravenously, subcutaneously, or intramuscularly.

19. The method of claim 17, wherein introducing the therapeutic amount of the cholinesterase inhibitor to the human occurs orally or transdermally.

20. The method of claim 17, wherein the therapeutic amount of the quaternary ammonium anti-cholinergic muscarinic receptor antagonist is introduced into the body of a human in a rapidly dissolving gelatin capsule, and the therapeutic amount of the acetyl-cholinesterase inhibitor is introduced into the body of a human in an enclosed acid resistant hydroxypropylmethylcellulose capsule configured for delayed release.

21. The method of claim 17, wherein the quaternary ammonium antimuscarinic compound is glycopyrrolate or trospium.
